(19) United States
(12) Patent Application Publication
(10) Pub. No.: US 2023/0218835 A1
(43) Pub. Date: Jul. 13, 2023

(54) MEDICAMENT DELIVERY DEVICE

(71) Applicant: SHL MEDICAL AG, ZUG (CH)

(72) Inventors: Munib Baber, Nacka Strand (SE); Magnus Gustafsson, Nacka Strand (SE); Stefan Lööf, Nacka Strand (SE)

(21) Appl. No.: 18/009,467

(22) PCT Filed: Jun. 15, 2021
(86) PCT No.: PCT/EP2021/066131

§ 371 Date: Dec. 9, 2022

(30) Foreign Application Priority Data
Jun. 30, 2020 (EP) 20183143.5

(51) Int. Cl. A61M 5/32; A61M 5/315

(52) U.S. Cl. CPC A61M 5/3245; A61M 5/3158

(57) ABSTRACT

A medicament delivery device comprising: a first component, and a second component arranged concentrically with the first component, wherein the second component is configured to move axially relative to the first component from a first position to a second position, wherein one of the first component and the second component has a radially extending flash, wherein the other one of the first component and the second component has a chamfer aligned with and facing the flash when the second component is in the first position, and wherein one of the chamfer and the flash is configured to contact and subsequently pass the other one of the chamfer and the flash when the second component is moved from the first position towards the second position to thereby facilitate movement of the second component.

### CROSS REFERENCE TO RELATED APPLICATIONS

The present application is a U.S. National Phase Application pursuant to 35 U.S.C. §371 of International Application No. PCT/EP2021/066131 filed Jun. 15, 2021, which claims priority to European Patent Application No. 20183143.5 filed Jun. 30, 2020. The entire disclosure contents of these applications are herewith incorporated by reference into the present application.

### TECHNICAL FIELD

The present disclosure generally relates to medicament delivery devices.

### BACKGROUND

Medicament delivery devices such as auto-injectors often comprise plastic components. The plastic components are usually injection moulded. The plastic components may have small defects, or flashes, as a result from the injection moulding. These small defects may interact with other components inside the medicament delivery device when the device is used.

Potential drawbacks with this are that the medicament delivery device may not work as intended or that some users with impaired finger capabilities and/or reduced strength may have difficulties to operate the medicament delivery device.

The manufacturing defects may be removed by machining, which however requires additional process steps.

### SUMMARY

An object of the present disclosure is to provide a medicament delivery device which solves, or at least mitigates problems of the prior art.

The presently disclosed concept is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

There is hence provided a medicament delivery device comprising: a first component, and a second component arranged concentrically with the first component, wherein the second component is configured to move axially relative to the first component from a first position to a second position, wherein one of the first component and the second component has a radially extending flash, wherein the other one of the first component and the second component has a chamfer aligned with and facing the flash when the second component is in the first position, and wherein one of the chamfer and the flash is configured to contact and subsequently pass the other one of the chamfer and the flash when the second component is moved from the first position towards the second position to thereby facilitate movement of the second component.

The chamfer hence facilitates for the second component to move axially, which would otherwise have been made more difficult due to the flash, for example because of an edge that is at right angle with the flash. The force required for moving the second component towards the second position will therefore be lower. Intended operation of the medicament delivery device may thereby be ensured and/or the operation of the medicament delivery device may be facilitated for users with impaired physical abilities.

The component of the first component and the second component which is provided with the flash may be an injection moulded component. The component of the first component and the second component which is provided with the flash may be made of plastic.

The medicament delivery device may be an injector such as an auto-injector, an inhaler or an eye dispenser.

The chamfer may be axially spaced apart from the flash when the second component is in the first position.

According to one embodiment the medicament delivery device has a proximal end and a rear end, wherein the chamfer tapers in a direction from the distal end towards the front end.

According to one embodiment the medical delivery device has a longitudinal central axis and wherein the chamfer has a chamfer surface that faces the longitudinal central axis.

The chamfer surface defines an inclined plane that intersects the longitudinal central axis of the medicament delivery device.

According to one embodiment the second component is biased towards the second position. The medicament delivery device may comprise a resilient member such as a spring configured to bias or urge the second component towards the second position.

According to one embodiment the first component is provided with the flash and the second component is arranged radially outside the first component, and wherein the flash extends radially outwards.

One embodiment comprises a housing, wherein the first component and the second component are arranged in the housing, and wherein the first position is a retracted position and the second position is an extended position relative to the housing, wherein in the first position the second component is retracted further into the housing than in the second position.

According to one embodiment the second component is a delivery member cover.

According to one embodiment the first component comprises a radial window, wherein the chamfer forms a radially inner rear edge of the radial window.

According to one embodiment the first component is a medicament container holder.

One embodiment comprises a trigger button, wherein the delivery member cover is configured to initially move from the second position to the first position, wherein the medicament container holder has a pivotable see-saw lever provided with a front see-saw arm and a rear see-saw arm, the rear see-saw arm being configured to block the trigger button from axial movement when the delivery member cover initially is in the second position, wherein the delivery member cover is configured to urge the rear see-saw arm radially inwards when the delivery member cover is moved from the second position towards the first position, enabling the trigger button to move forward axially to a depressed position.

According to one embodiment the flash is arranged on the front see-saw arm.

According to one embodiment the flash is arranged on the rear see-saw arm.

According to one embodiment the second component is a trigger button.

One embodiment provides a medicament delivery device comprising: a medicament delivery device housing, and a trigger button arranged concentrically with the medicament delivery device housing, wherein the medicament delivery device housing and the trigger button are slidably interacting to each other, wherein the trigger button is configured to move axially relative to the medicament delivery device housing from an initial position to a pressed position, wherein one of the medicament delivery device housing and the trigger button has a radially extending flash, wherein the other one of the medicament delivery device housing and the trigger button has a chamfer aligned with and facing the flash when the trigger button is in the initial position of the trigger button, and wherein one of the chamfer and the flash is configured to contact and subsequently pass the other one of the chamfer and the flash when the trigger button is moved from the initial position towards the pressed position to thereby facilitate movement of the trigger button.

The trigger button is at least partially received within the medicament delivery device housing.

One embodiment provides a medicament delivery device comprising: a medicament delivery device housing, and a medicament container holder arranged concentrically with the medicament delivery device housing, wherein the medicament delivery device housing and the medicament container holder are slidably interacting to each other, wherein the medicament container holder is configured to move axially relative to the medicament delivery device housing from a first position to a second position, wherein one of the medicament delivery device housing and the medicament container holder has a radially extending flash, wherein the other one of the medicament delivery device housing and the medicament container holder has a chamfer aligned with and facing the flash when the medicament container carrier is in the first position of the medicament container holder, and wherein one of the chamfer and the flash is configured to contact and subsequently pass the other one of the chamfer and the flash when the medicament container holder is moved from the first position towards the second position to thereby facilitate movement of the medicament container holder.

The medicament container holder is received within the medicament delivery device housing.

According to one embodiment, the movement of the medicament container holder is moved from the first position towards the second position is configured to move the medicament container holder together with the medicament container in the proximal direction.

According to one embodiment, when the medicament delivery device is an injection device, the movement of the medicament container holder is moved from the first position towards the second position is configured to perform an auto needle insertion operation.

According to one embodiment, the movement of the medicament container holder is moved from the first position towards the second position is configured to move the medicament container holder together with the medicament container in the distal direction.

According to one embodiment, when the medicament delivery device is an injection device, the movement of the medicament container holder is moved from the first position towards the second position is configured to perform an auto needle retraction operation.

According to one embodiment, the first component and the second component are slidably interacting to each other.

According to one embodiment, the first component and the second component are movable, but irremovable.

According to one embodiment the flash extends radially at most 0.1 mm.

Generally, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

The specific embodiments of the presently disclosed concept will now be described, by way of example, with reference to the accompanying drawings, in which:

FIG. 1 schematically shows a first example of a medicament delivery device;

FIG. 2 schematically shows a detail of a longitudinal section of the medicament delivery device FIG. 1 when a second component is in a first position;

FIG. 3 shows the second component moving towards a second position;

FIG. 4 is a perspective view of an example of a medicament delivery device;

FIG. 5 shows the medicament delivery device in FIG. 4 without its housing;

FIG. 6 is a longitudinal section of a portion of the medicament delivery device in FIG. 5;

FIG. 7 shows the medicament delivery device in FIG. 5 when the delivery member cover has been moved to a retracted position;

FIG. 8 shows the medicament delivery device in FIG. 5 after activation;

FIG. 9 shows a close-up view after activation; and

FIG. 10 shows the medicament delivery device in a locked-out state after use.

### DETAILED DESCRIPTION

The presently disclosed concept will now be described more fully hereinafter with reference to the accompanying drawings, in which exemplifying embodiments are shown. The presently disclosed concept may, however, be embodied in many different forms and should not be construed as limited to the embodiments set forth herein; rather, these embodiments are provided by way of example so that this disclosure will be thorough and complete, and will fully convey the scope of the presently disclosed concept to those skilled in the art. Like numbers refer to like elements throughout the description.

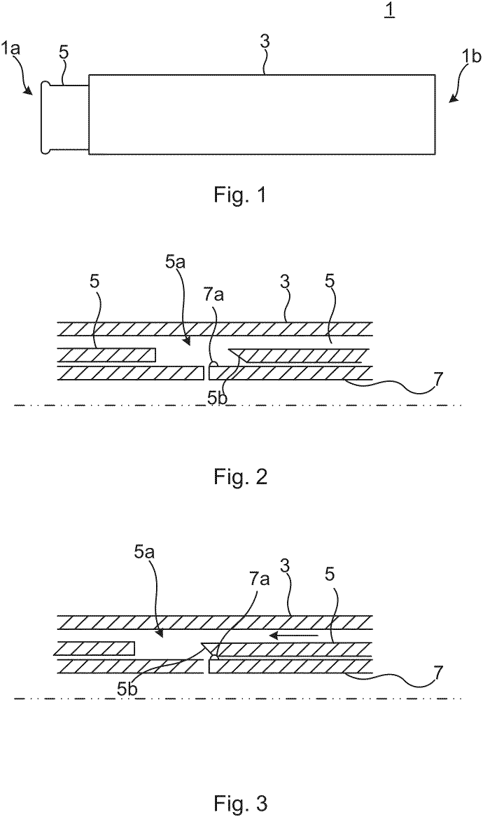

FIG. 1 schematically shows an example of a medicament delivery device 1. The medicament delivery device may for example be an injector, such an auto-injector, an inhaler or an eye dispenser.

The exemplified medicament delivery device 1 comprises a housing 3 and a delivery member cover 5 arranged in the housing 3. The delivery member cover 5 is arranged concentrically with the housing 3.

The medicament delivery device 1 has a proximal end 1a and a distal end 1b. The delivery member cover 5 has a front-end portion that protrudes from the housing 3. The front-end portion forms the proximal end 1a of the medicament delivery device 1.

The delivery member cover 5 is configured to move linearly relative to the housing 3. The delivery member cover 5 is configured to move along the axial direction of the housing 3. The delivery member cover 5 is configured to be moved between an extended position relative to the housing 3, shown in FIG. 1, and a retracted position. The medicament delivery device 1 may have a delivery member, such as a needle, which is covered by the delivery member cover 5 in the extended position and exposed when the delivery member cover 5 is in the retracted position. The retracted position is herein also referred to as the “first position”. The extended position is herein also referred to as the “second position”. The delivery member cover 5 is biased towards the extended position.

The delivery member cover 5 may be moved from the extended position, or second position, to the retracted position, or first position to activate the medicament delivery device 1 or to enable activation of the medicament delivery device 1, depending on the specific realisation of the medicament delivery device.

The delivery member cover 5 is typically moved from the extended position to the retracted position by pushing the delivery member cover 5 towards the injection site. The delivery member is thereby exposed and may penetrate the site of injection in case the delivery member is a needle.

When the medicament delivery device 1 is moved away from the injection site, the delivery member cover 5 is returned to the extended position. This is because the delivery member cover 5 is biased, or urged, towards the extended position.

At least some of the components of the medicament delivery device 1 may be manufactured by injection moulding. Injection moulding typically provides the moulded component with flashes after the component has been removed from the mould. The flashes are normally located at the edges of an injection moulded component and/or in regions extending between two mould halves.

FIG. 2 shows a portion of a longitudinal section of a portion of one example of the medicament delivery device 1.

The delivery member cover 5 is in FIG. 2 arranged in the retracted position, i.e. in the first position.

The medicament delivery member 1 comprises a medicament container holder 7. The medicament container holder 7 is arranged radially inwards of the delivery member cover 5. The medicament container holder 7 is provided with a radially outwards extending flash 7a. The flash 7a is located at an edge of the medicament container holder 7.

The medicament container holder 7 is an example of a “first component” as used herein. The delivery member cover 5 is an example of a “second component” as used herein.

The delivery member cover 5 has a radial window 5a. The delivery member cover 5 has a chamfer 5b. The chamfer 5b forms a radially inner rear edge of the radial window 5a. The chamfer 5b is hence located at the distal end of the radial window 5a, on the inner side of the delivery member cover 5. The inner side faces the medicament container holder 7. The chamfer 5b is tapering in a direction from the distal end 1b towards the proximal end 1a.

In the retracted position of the delivery member cover 5, the chamfer 5b is arranged axially closer to the distal end 1b of the medicament delivery device 1 than the flash 7a. In the retracted position of the delivery member cover 5, the chamfer 5b faces the flash 7a. The chamfer 5b is axially aligned with the flash 7a.

FIG. 3 schematically shows the medicament delivery device 1 when the delivery member cover 5 is moved in the forward direction from the retracted position towards the extended position, i.e. from the first position towards the second position.

The chamfer 5b of the delivery member cover 5 contacts and subsequently moves past the flash 7a. Due to the chamfer 5b, movement of the inner rear edge of the radial window 5a past the flash 7a is facilitated. Thus, less force is required for moving the delivery member cover 5 back to the extended position.

The delivery member cover 5 may be configured to lock out when it reaches the extended position. The delivery member cover 5 is prevented from moving towards the retracted position when it has been locked out. There are a great number of different ways accomplish this as would be known to the person skilled in the art and will therefore not be described any further with respect to this example.

Because of the chamfer 5b it can be ensured that the delivery member cover 5 is moved back to the extended position from the retracted position when the medicament delivery device 1 is removed from the site of injection. It can thus be ensured that the delivery member cover 5 is locked out in the extended position. As a result, the safety of the used medicament delivery device 1 may be improved.

According to a variation of the medicament delivery device 1, the delivery member cover could instead or additionally be provided with the flash. For example, the housing could be provided with a radial window having a chamfer and the flash could be arranged radially outwards facing towards the chamfer of the housing when the delivery member cover is in the retracted position. Alternatively, the housing could have an edge provided with the flash and the delivery member cover could be provided with the chamfer facing the flash when the delivery member cover is in the retracted position. According to yet another variation, the medicament container holder could be provided with the chamfer and the delivery member cover could be provided with the flash. Variations are also envisaged where the flash and the chamfer interact when the delivery member cover is moved from the extended position towards the retracted position. In general, any two components that are arranged adjacent to each other and movable relative to each other may be provided with a respective one of the flash and the chamfer, with the chamfer being configured to facilitate movement between the two components due to the flash.

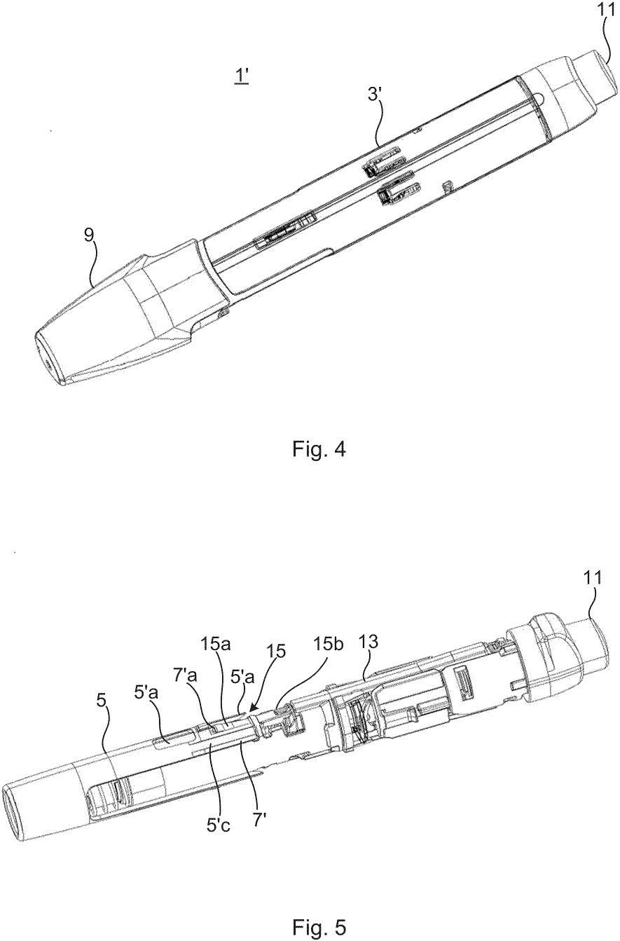

FIG. 4 shows a perspective view of one example of a medicament delivery device. The medicament delivery device 1′ comprises a housing 3′. The medicament delivery device 1′ comprises a cap 9 which can be attached to the proximal end of the housing 3′. The cap 9 is removable from the housing 3′. The cap 9 is to be removed from the housing 3′ before the medicament delivery device 1′ is to be used.

The medicament delivery device 1′ comprises a triggering button 11. The triggering button 11 is in the present example provided at a distal end of the medicament delivery device 1′. The triggering button 11 is configured to be moved parallel with the longitudinal central axis of the medicament delivery device 1′. The triggering button 11 is configured to be moved from an initial position shown in FIG. 4 to a depressed position to activate medicament expulsion.

FIG. 5 shows the medicament delivery device 1′ with the cap 9 and the housing 3′ removed. The medicament delivery device 1′ comprises a delivery member cover 5′. The delivery member cover 5′ is an example of a “second component” as used herein. The delivery member cover 5′ is movable relative to the housing 3 just like the delivery member cover 5 described earlier.

The medicament deliver device 1′ comprises a medicament container holder 7′. The medicament container holder 7′ is configured to hold a medicament container or syringe. The medicament container holder 7′ is an example of a “first component” as used herein. The medicament container holder 7′ is arranged concentrically with the delivery member cover 5. The delivery member cover 5 is arranged radially outside of the medicament container holder 7′.

The medicament container holder 7′ has a pivotable see-saw lever 15. The see-saw lever 15 is pivotably about an axis that is transverse or perpendicular relative to the longitudinal central axis of the medicament delivery device 1′. The see-saw lever 15 is provided on an outer side of the medicament container holder 7′.

The see-saw lever 15 has a front see-saw arm 15a and a rear see-saw arm 15b. The front see-saw arm 15a and the rear see-saw arm 15b are pivotable about a fulcrum which connects the two arms 15a and 15b. The front see-saw arm 15a is provided with a flash 7′a. The flash 7′a extends radially outwards from the front see-saw arm 15a.

The delivery member cover 5′ has parallel rearwards extending legs 5′c. The delivery member cover 5′ has a space between the legs 5′c. The see-saw lever 15 is arranged in the space between two legs 5′c. In particular, the front see-saw arm 15a is arranged in the space when the delivery member cover 5′ is in the extended position.

The delivery member cover 5′ has a radial window 5′a arranged axially aligned with the space. The radial window 5′a is arranged closer to the proximal end of the delivery member cover 5′a than the space. The space is hence arranged rearwards relative to the radial window 5′a.

The medicament delivery device 1′ comprises an elongated blocking structure 13 configured to move concurrently with the triggering button 11. The blocking structure 13 and the triggering button may be formed integral or as different components. The blocking structure 13 may be in the form of an arm.

The blocking structure 13 extends along the axial direction of the medicament delivery device 1′. The blocking structure 13 extends towards a distal end face of the rear see-saw arm 15b when the delivery member cover 5′ is in the extended position. The distal end face of the rear see-saw arm 15b prevents the blocking structure 13 and thus the triggering button 11 from being moved forward. The triggering button 11 is thus maintained in the initial position when the delivery member cover 5′ is in the extended position.

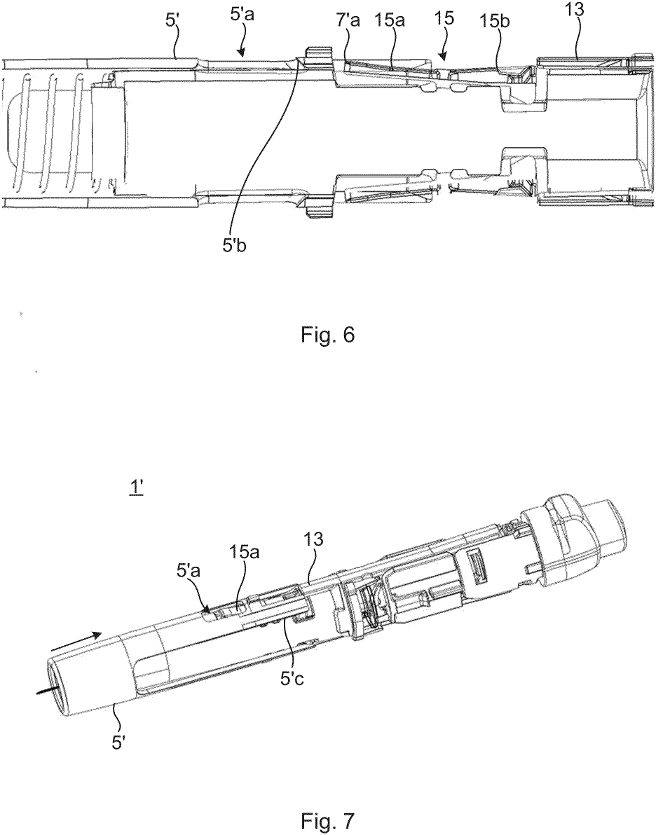

FIG. 6 shows a longitudinal section of a portion of the medicament delivery device 1′ with the housing 3′ removed.

The radial window 5′a has a radially inner rear edge which is chamfered. The delivery member cover 5′ hence has a chamfer 5′b. This configuration is like in the example of FIGS. 1-3.

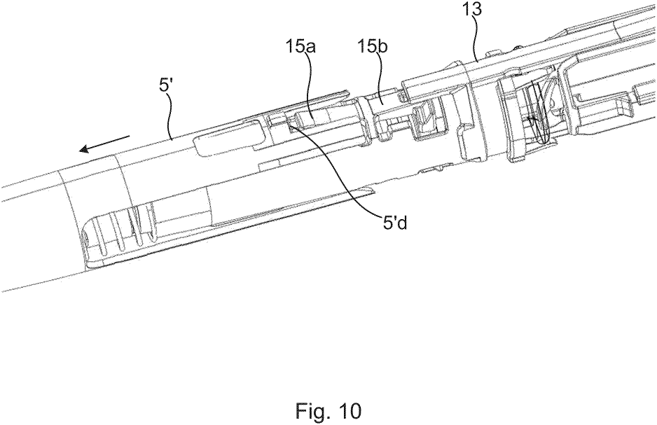

The interaction between the flash 7′a and the chamfer 5′b during operation of the medicament delivery device 1′ will now be described in more detail with reference to FIGS. 7-10.

FIG. 7 illustrates when the delivery member cover 5′ has been moved from the extended position towards the retracted position. The front see-saw arm 15a is moved into the radial window 5′a. The legs 5′c of the delivery member cover 5′ move radially over the rear see-saw arm 15b and urge the rear see-saw arm 15b radially inwards. The blocking structure 13 is thereby able to move forward because the distal end face of the rear see-saw arm 15b has been moved to a radial level below the blocking structure 13.

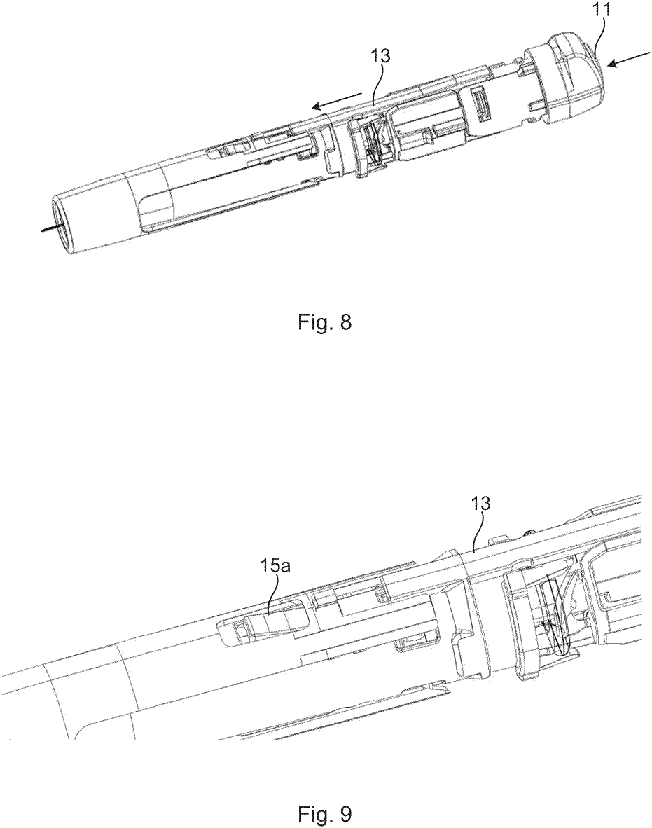

FIG. 8 shows when the trigger button 11 has been depressed and set in the depressed position. The blocking structure 13 is moved forward as the trigger button 11 is moved forward. This motion activates the power pack of the medicament delivery device 1′. Medicament expulsion is thereby performed. The operation of the power pack is not a topic of this disclosure and will therefore not be disclosed in any more detail herein.

FIG. 9 is a close-up view of the state of the medicament delivery device 1′ shown in FIG. 8.

FIG. 9 shows when the delivery member cover 5′ has been released from the retracted position and has returned to the extended position. Due to the chamfer 5′b shown in FIG. 6 the delivery member cover 5′ can easily move past the flash 6′a of the front see-saw arm 15a as the delivery member cover 5′ is moved back to the extended position.

The blocking structure 13 is maintained in its forward position obtained when the trigger button 11 was depressed. The rear see-saw arm 15b is thus pressed radially inwards by the blocking structure 13 as shown. Due to the lever functionality, the front see-saw arm 15a is therefore arranged in a radially more outwards extending position than before the blocking structure 13 pressed the rear see-saw arm 15b radially inwards. The front see-saw arm 15a thus blocks a distal end 5′d from moving rearwards. The delivery member cover 5′ is thus prevented from moving rearwards. The delivery member cover 5′ has thereby been locked out.

The presently disclosed concept has mainly been described above with reference to a few examples. However, as is readily appreciated by a person skilled in the art, other embodiments than the ones disclosed above are equally possible within the scope of the presently disclosed concept, as defined by the appended claims.

1-15. (canceled)

16. A medicament delivery device comprising:
a first component, and
a second component arranged concentrically with the first component,
wherein the second component is configured to move axially relative to the first component from a first position to a second position,
wherein one of the first component and the second component has a radially extending flash,
wherein the other one of the first component and the second component has a chamfer aligned with and facing the flash when the second component is in the first position, and
wherein one of the chamfer and the flash is configured to contact and subsequently pass the other one of the chamfer and the flash when the second component is moved from the first position towards the second position to thereby facilitate movement of the second component.

17. The medical delivery device as claimed in claim 16, wherein the medicament delivery device has a proximal end and a distal end, wherein the chamfer tapers in a direction from the distal end towards the proximal end.

18. The medicament delivery device as claimed in claim 16, wherein the medical delivery device has a longitudinal central axis and wherein the chamfer has a chamfer surface that faces the longitudinal central axis.

19. The medicament delivery device as claimed in claim 16, wherein the second component is biased towards the second position.

20. The medicament delivery device as claimed in claim 16, wherein the first component is provided with the flash and the second component is arranged radially outside the first component, and wherein the flash extends radially outwards.

21. The medicament delivery device as claimed in claim 16, wherein the second component is a delivery member cover or a trigger button or a medicament container holder.

22. The medicament delivery device as claimed in claim 16, comprising a housing.

23. The medicament delivery device as claimed in claim 21 further comprising a housing , wherein when the second component is a delivery member cover or a trigger button, , wherein the first component and the second component are arranged in the housing, and wherein the first position is a retracted position and the second position is an extended position relative to the housing, wherein in the first position the second component is retracted further into the housing than in the second position.

24. The medicament delivery device as claimed in claim 23, when the second component is a delivery member cover, wherein the second component comprises a radial window, wherein the chamfer forms a radially inner rear edge of the radial window.

25. The medicament delivery device as claimed in claim 23, dependent on claim 6, when the second component is a delivery member cover or a trigger button, wherein the first component is a medicament container holder.

26. The medicament delivery device as claimed in claim 25, when the second component is a delivery member cover, the medicament delivery device comprising a trigger button, wherein the delivery member cover is configured to initially move from the second position to the first position, wherein the medicament container holder has a pivotable see-saw lever provided with a front see-saw arm and a rear see-saw arm, the rear see-saw arm being configured to block the trigger button from axial movement when the delivery member cover initially is in the second position, wherein the delivery member cover is configured to urge the rear see-saw arm radially inwards when the delivery member cover is moved from the second position towards the first position, enabling the trigger button to move forward axially to a depressed position.

27. The medicament delivery device as claimed in claim 26, wherein the flash (7′a) is arranged on the front see-saw arm.

28. The medicament delivery device as claimed in claim 16, wherein the flash extends radially at most 0.1 mm.

29. The medicament delivery device as claimed in claim 16, wherein the first component and the second component are slidably interacting to each other.

30. The medicament delivery device as claimed in claim 16, wherein the first component and the second component are movable, but irremovable.

31. A medicament delivery device comprising:
a housing having a longitudinal axis;
a first sleeve positioned within the housing; and
a second sleeve arranged concentrically and slidably with the first sleeve such that the first and second sleeves interact with each other when the second sleeve moves axially relative to the first sleeve from a first position to a second position,
wherein one of the first sleeve and the second sleeve comprises a flash that extends radially relative to the longitudinal axis,
wherein the other one of the first sleeve and the second sleeve has a chamfer aligned with and facing the flash when the second sleeve is in the first position, and
wherein one of the chamfer and the flash contacts and subsequently moves axially past the other one of the chamfer and the flash when the second moves from the first position towards the second position.

32. The delivery device of claim 31, wherein the chamfer is on the second sleeve and has a chamfer surface that faces the longitudinal central axis.

33. The delivery device of claim 31, wherein the flash is on the first sleeve and extends radially outward relative to the longitudinal axis.

34. The delivery device of claim 31, wherein first sleeve further comprises a a pivotable see-saw lever comprising a front see-saw arm and a rear see-saw arm, where the flash is arranged on the front see-saw arm.

35. The delivery device of claim 34 further comprising a trigger button, where the rear see-saw arm is configured to block the trigger button from axial movement when the second sleeve is initially in the second position, where the second sleeve is configured to urge the rear see-saw arm radially inwards when the second sleeve is moved from the second position towards the first position, enabling the trigger button to move forward axially to a depressed position.
